(19) United States
(12) Patent Application Publication
(10) Pub. No.: US 2019/0298164 A1
(43) Pub. Date: Oct. 3, 2019

(54) FINGER-MOUNTED VIDEO LARYNGOSCOPE AND METHOD OF USE

(71) Applicant: Ariel Daube, Brooklyn, NY (US)

(72) Inventor: Ariel Daube, Brooklyn, NY (US)

(21) Appl. No.: 16/364,791

(22) Filed: Mar. 26, 2019

Related U.S. Application Data
(60) Provisional application No. 62/649,076, filed on Mar. 28, 2018.

(51) Int. Cl. A61B 1/267; A61M 16/04; A61B 1/06

(52) U.S. Cl. CPC A61B 1/267; A61B 1/04; A61B 1/0684; A61M 16/0488

(57) ABSTRACT

A finger-mounted video laryngoscope is disclosed. The system includes an endoscopic camera attached to the tip of a finger condom. The camera is connected to a hollow tube that acts as a conduit through which an endotracheal tube introducer can be passed. The camera and introducer conduit are angled toward the patient's larynx once the operator palpates the patient's epiglottis. Appropriate positioning is confirmed by visualizing the larynx on the screen.

### RELATED APPLICATION

This application claims the benefit of priority from U.S. Provisional Application No. 62/649,076, filed on Mar. 28, 2018, the entirety of which is incorporated by reference.

### FIELD OF THE INVENTION

This invention relates generally to the field of endotracheal intubation and more specifically to video-assisted intubation.

### BACKGROUND OF THE INVENTION

Endotracheal intubation, the procedure of placing a flexible plastic tube in a patient's trachea, is performed to allow for optimal mechanical ventilation of the patient. Intubation is indicated during many circumstances including general anesthesia, resuscitation of a patient with respiratory failure and when a patient's airway patency is compromised.

Failure to successfully intubate a patient in a timely manner can result in severe oxygen deprivation, causing permanent brain damage and sometimes death. Thus, the optimal intubation technique should have as high a success rate as possible.

Most often, intubation is performed by means of laryngoscopy; i.e., visualization of the larynx. The larynx comprises the vocal cords, which are located at the entrance of the trachea. Once the vocal cords are visualized, an endotracheal tube is passed between them and into the trachea.

Laryngoscopy can be accomplished by a number of means. The most commonly utilized prior art method is direct laryngoscopy. This is performed by introducing a rigid blade into the patient's mouth in order to lift the tongue and airway structures anteriorly to allow for a direct line of site into the larynx. This method has the following limitations: It is difficult to perform and has an unacceptably high failure rate among novice providers; the blade has a fixed geometry and thus does not provide an optimal view for patients with difficult anatomy; and it utilizes a rigid blade, which can damage the teeth and oral/pharyngeal structures.

The second most commonly utilized prior art method is video laryngoscopy, whereby a camera is attached to the tip of a laryngoscope blade, allowing for indirect visualization of the larynx. Generally, this method allows for easier visualization than direct laryngoscopy. However, this method is still relatively difficult to perform among novice providers because it utilizes fixed geometry blades which cannot adapt to unique anatomy; and it still carries a risk of damaging the oral and pharyngeal structures since it uses a rigid blade.

A third prior art method for intubating a patient is using fiberoptic endoscopy. In this technique, an articulating fiberscope is advanced through the patient's mouth or nose into the trachea. An endotracheal tube, which has been pre-loaded onto the fiberscope shaft, is then railroaded over the scope into the trachea. This technique can readily adapt to patients with difficult anatomy.

However, mastering the articulation of an endoscope is difficult, and it can only be performed reliably by those with extensive experience.

A fourth, rarely performed, prior art method is known as blind digital (finger) intubation. In this technique, the operator's index and middle fingers are inserted into the patient's mouth until the epiglottis, a cartilaginous structure at the base of the tongue, is palpated. Subsequently, an endotracheal tube is introduced into the mouth and directed along the posterior surface of the epiglottis in the hopes that the endotracheal tube enters the trachea. This technique is limited by lack of real-time visual feedback on the path of the endotracheal tube, placing the patient at risk for esophageal intubation and glottic tissue injury. Additionally, achieving the dexterity necessary to guide the endotracheal tube into the trachea is difficult.

### OBJECTS AND SUMMARY OF THE INVENTION

It is an object of this invention to improve the technique of digital (finger) intubation by adding real-time visual feedback.

It is an object of this invention to improve the technique of digital (finger) intubation by utilizing a conduit for an endotracheal tube introducer that is angled preferentially toward the patient's trachea.

It is an object of this invention to add tactile feedback for the operator to guide the laryngoscope into the appropriate position.

It is an object of this invention to allow for versatile manipulation of the laryngoscope by means of flexion/extension and advancement/retraction of the finger.

It is an object of this invention to minimize the amount of manipulation needed by optimizing the angle between the laryngoscope and the operator's distal phalanx.

It is an object of this invention to minimize the risk of injury to the oral and pharyngeal structures.

It is an object of this invention to be highly portable.

To this end, the present arrangement provides for a medical device having a laryngoscope, where the laryngoscope has a finger portion, a camera, and an introducer conduit. The camera and the introducer conduit are perpendicular to the finger portion and parallel to one another.

### BRIEF DESCRIPTION OF THE DRAWINGS

The present arrangements can be best understood through the following description and accompanying drawings, wherein

FIG. 1 depicts the parallel relationship of the camera to the introducer conduit. Additionally, it shows how the proximal end of the introducer slit is wider than the distal end.

FIG. 2 shows the geometry of the aspects of the laryngoscope that interface with the operator's finger.

FIG. 3 is a view of the camera lens and surrounding LED lights. It also depicts the relationship between the camera lens and the distal end of the introducer conduit.

FIG. 4 illustrates how the finger-mounted video laryngoscope is worn right before it is placed in the patient's mouth. Depicted are the finger condom and laryngoscope on the operator's finger. The endotracheal tube introducer is loaded into the introducer conduit. The laryngoscope is connected to a screen module.

FIG. 5 is a sagittal cross-sectional view of patient's mouth/pharynx/larynx with the introducer having been passed into the trachea. The finger pad is shown palpating the epiglottis.

FIG. 6 shows a possible embodiment in which the laryngoscope is affixed to the user's finger by means of an exoskeleton.

### DETAILED DESCRIPTION

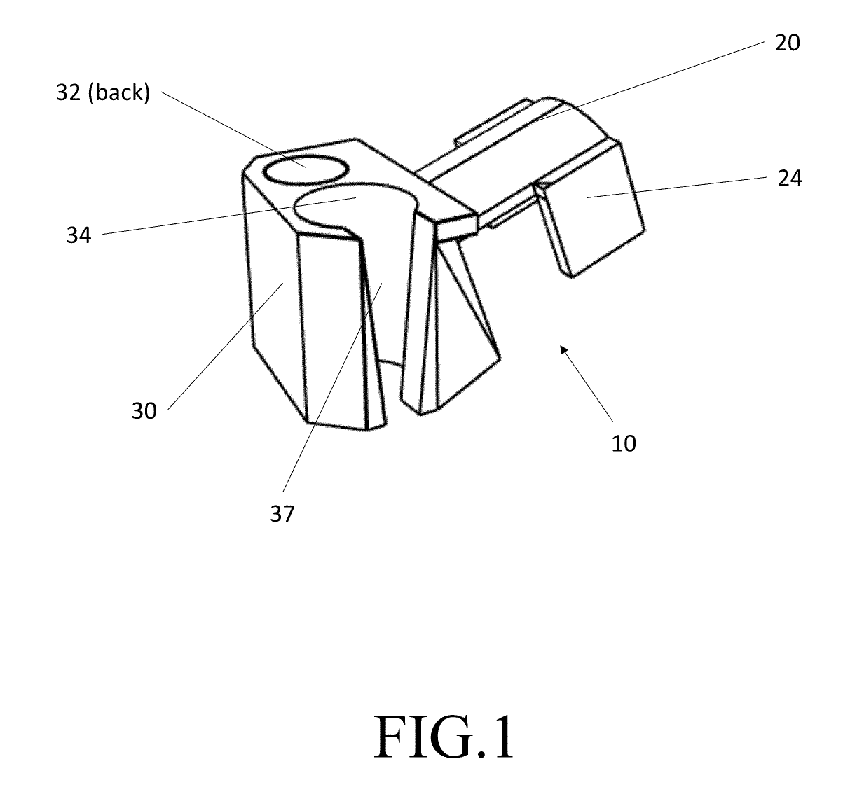

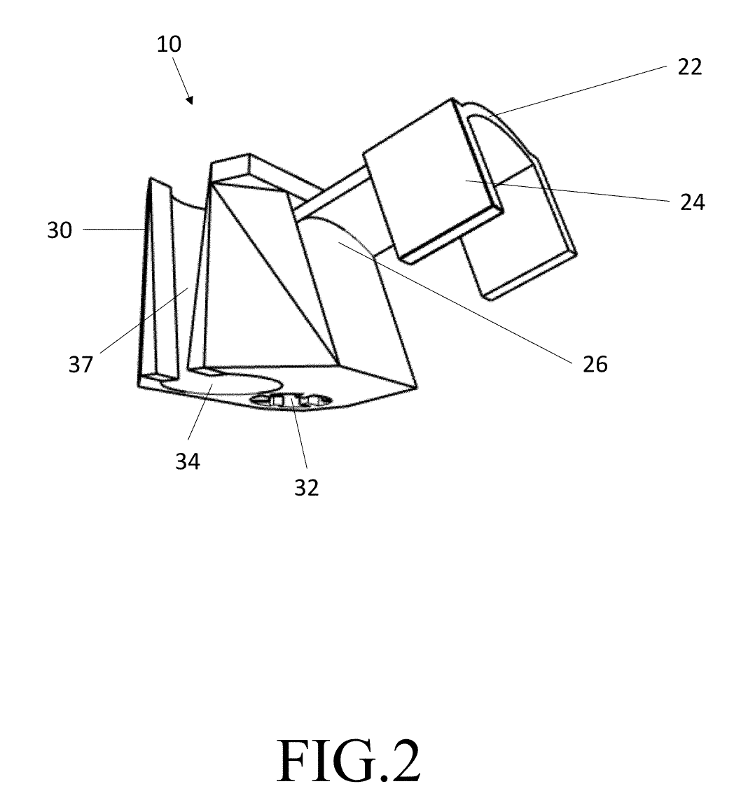

In one embodiment of the present invention as illustrated in FIGS. 1-3 a finger-mounted video laryngoscope 10 is provided. Laryngoscope 10 has a finger portion 20 and a scope portion 30. As shown in FIG. 2, finger portion 20 has a curved fingernail portion 22, two flexible finger tabs 24 and a recessed area 26 for a finger-tip of the wearer.

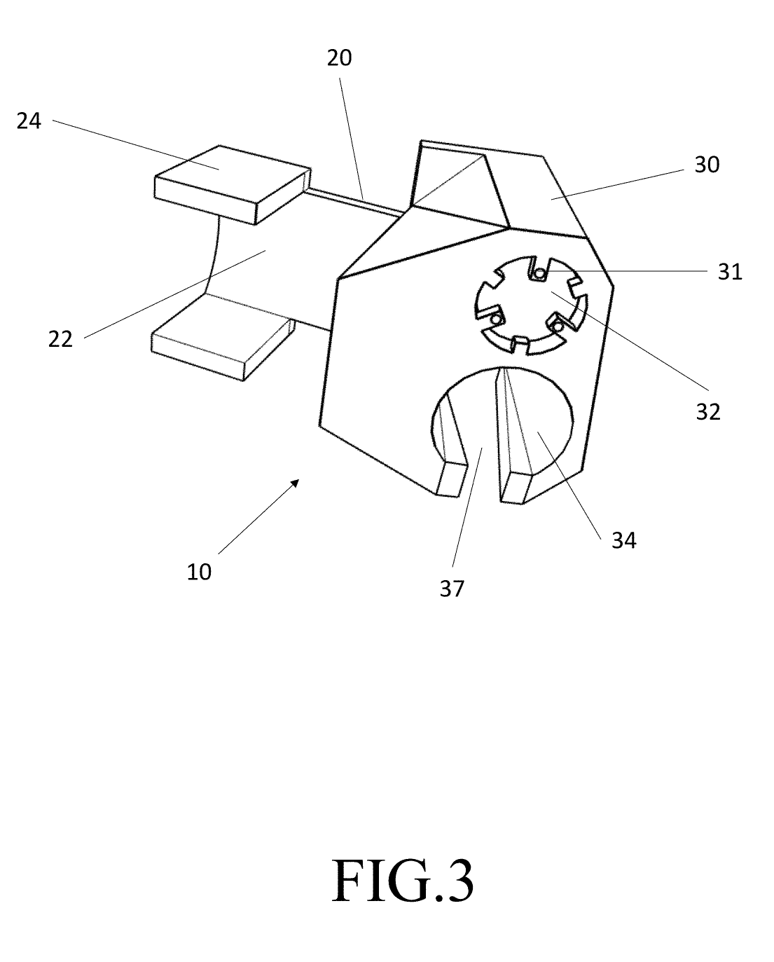

Regarding the scope portion, there is a camera 32, and an introducer conduit 34. As shown in FIG. 3, the camera 32, at the bottom opening that will face downward towards the vocal cords, may be fitted with peripheral lighting such as LED lights 31.

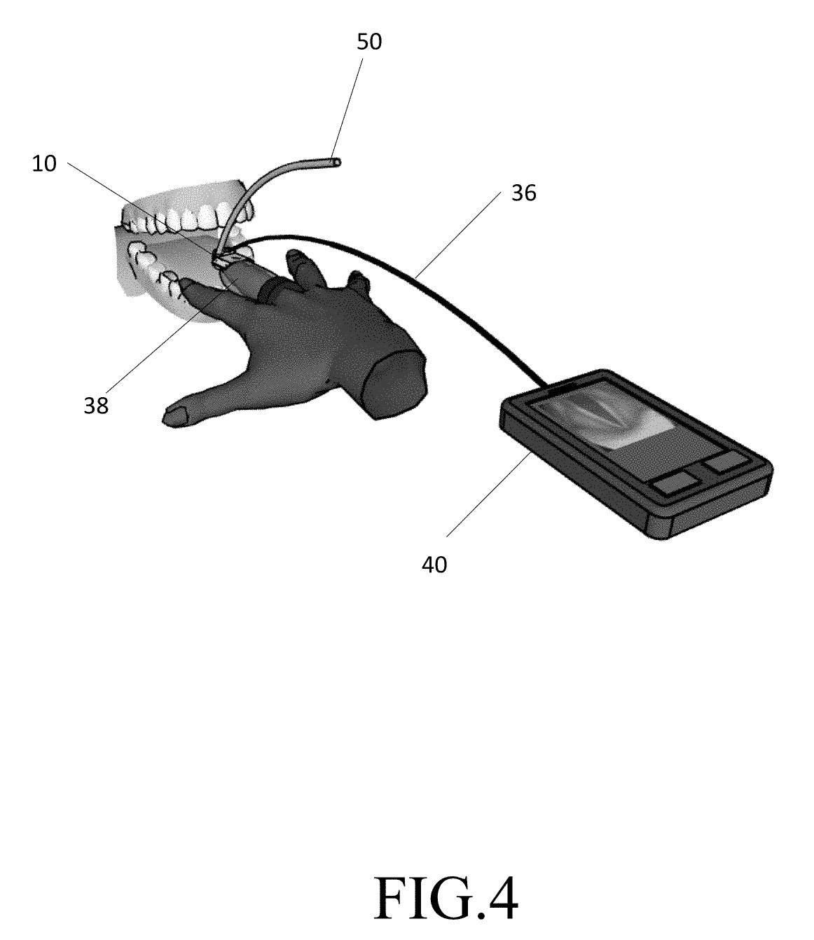

As shown in FIG. 4, laryngoscope 10 is shown attached to a user's finger. Camera 32 and LED lights 31 are coupled to an electronic device 40 via a cord 36 so that the user can view the camera output on the screen of electronic device 40. In FIG. 4, an introducer 50 is attached to laryngoscope 10 in order to provide a guide over which the endotracheal tube to be inserted into the patient.

As shown in FIG. 4, in one embodiment, endoscopic camera 32 is attached to the tip of a finger condom 38 such that once finger condom 38 is donned with laryngoscope 10, the angle between the camera's axis and the distal phalanx would be roughly 110 degrees. This allows for optimal visualization of the larynx when the operator's finger pad palpates the epiglottis. However, in other embodiments, this angle between the camera's axis and the distal phalanx might be modified by those skilled in the art to improve the effectiveness of the view, possibly for different sized patients or patients with particular issues that affect endotracheal intubation. In some embodiments, the angle may range from below 90 degrees to 180 degrees, possibly for patients of different size or age. In one embodiment, a lens of camera 32 may be surrounded by LED light bulbs 31 to illuminate the field of view. These bulbs 31 could be infrared for use in low-light tactical situations.

In one embodiment, camera 32 is connected to tubular conduit through which endotracheal tube introducer 50 could be passed. The axis of the distal end of the introducer conduit 34 and the axis of the camera's focus is parallel such that when the larynx is visualized on camera 32, introducer conduit 34 would point directly toward the larynx.

In one embodiment, the geometry of introducer conduit 34, shown for example in FIGS. 1-3, is such that it maintains a moderate degree of friction with introducer 50 so that introducer 50 could only be advanced/retracted intentionally.

This friction could be generated by a number of potential embodiments:

In one embodiment, the internal diameter of introducer conduit 34 would be only slightly larger than the outer diameter of introducer 50 itself If a narrower introducer 50 were to be used such as for pediatric intubations, an attachment could be inserted into introducer conduit 34 to narrow its internal diameter.

In an alternative embodiment, the appropriate degree of friction could be achieved by a curved introducer conduit 34 such that the rigidity of introducer 50 maintains a normal force with the internal wall of introducer conduit 34.

In another embodiment, introducer conduit 34 has a flexible diaphragm or flexible tabs that could mold to different diameter introducers 50.

In all of three of these embodiments, lubrications may or may not be applied depending on the desired amount of friction.

In one embodiment, as shown in FIG. 1-3, introducer conduit 34 has a slit 37 on its side such that a lateral force on introducer 50 would dislodge it from conduit 34. Slit 37 may be narrow enough to prevent inadvertent dislodgement of introducer 50 from conduit 34, however loose enough that it would take a reasonably small amount of force to dislodge introducer 50 from conduit 34. The proximal end of slit 37 would have a wider aperture than the distal end to facilitate initiation of the dislodgement.

In one embodiment, camera 32 and conduit 34 encasement would be attached to finger condom 38 in such a way that flexion/extension of the distal phalanx would result in similar and parallel movement of the encasement/laryngoscope 10. To restrict movement of the encasement 10 that is independent from movement of the distal phalanx, the aspect of the encasement 10 that interfaces with finger-tip portion 26 would most likely be recessed to create a snug fit with the user's finger-tip. Likewise, the aspect that interfaces with the fingernail is a curved fingernail portion 22 to roughly match the curvature of the finger. Furthermore, encasement 10 is connected to semi-rigid elastic tabs 24 that sandwich and squeeze the lateral aspects of the distal phalanx. Additional tabs might connect the encasement to the finger condom 38 to improve adhesion of the two elements.

So as to maintain a narrow profile to accommodate patients with limited mouth opening (e.g. pediatric patients or those with trismus), the dimensions of laryngoscope encasement 10 extend as minimally as possible beyond the dorsal and ventral aspects of the finger. Encasement 10 might extend past the fingertip, i.e. extend the finger's length, so as to accommodate jaws that are relatively large compared to the operator's finger length. Encasement 10 may be made from a material such as silicone or soft plastic, and its edges would be softened in order to minimize the risk of scratching the patient's mucous membranes.

In one embodiment, endotracheal tube introducer 50 is made of material flexible enough to allow for sufficient bending to make a sharp enough angle when it passes from the mouth into introducer conduit 34. However, inducer 50 is rigid enough to prevent kinking. Additionally, introducer 50 may have a hollow core to facilitate insufflation of oxygen and suctioning of secretions. In another embodiment, endotracheal tube introducer 50 could have an additional endoscopic camera (other than camera 32) attached to its tip to aid with visualization of the larynx and confirmation of tracheal placement.

In one embodiment, finger condom 38 is made from flexible material such as nitrile or silicone. Overall, finger condom 38 is thick enough to prevent tearing. However, the area over the finger pad would be thinner than the rest of finger condom 38 so as to maximize tactile sensation. The dimensions of finger condom 38 are such its elasticity accommodates larger diameter fingers comfortably while maintaining a tight grip on narrower fingers so as not to slip off during the procedure. Finger condom 38 might be textured on the inside and outside to allow for improved grip with the operator's glove and the patient's mucous membranes, respectively. The length of finger condom 38 is adjustable by the degree to which it is unfurled to reach the base of the finger.

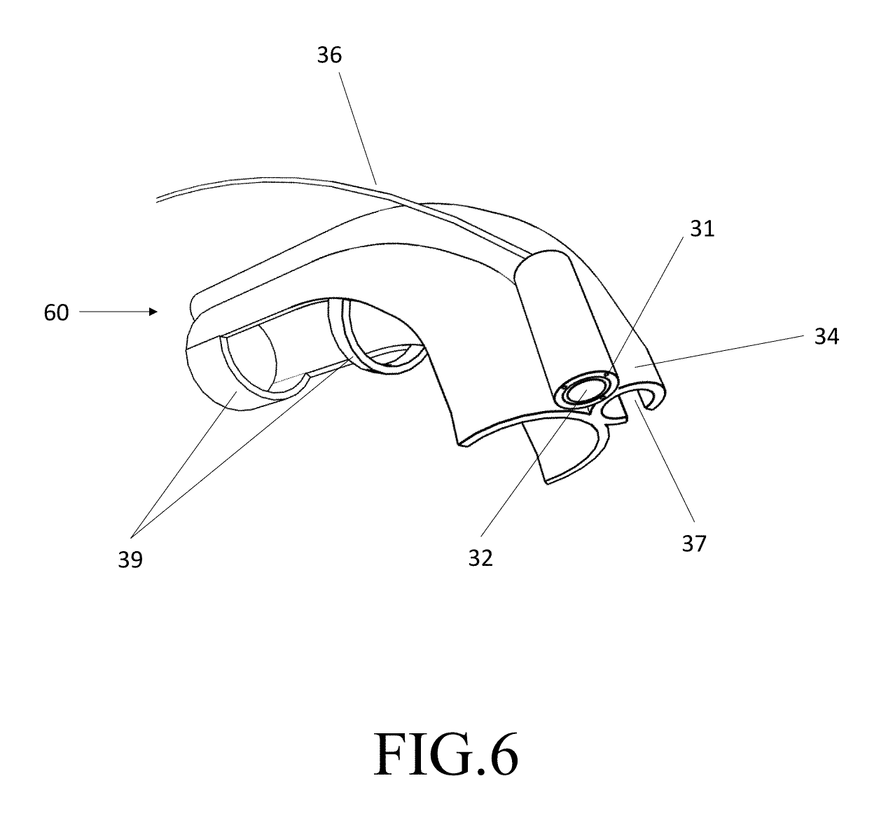

In an alternative embodiment, finger-mounted laryngoscope 10 could attach to the operator's finger by means of an exoskeleton 60 rather than finger condom 38. Exoskeleton 60 might be made from semi-rigid plastic or silicone and its edges softened to minimize the risk of injury to the mucous membranes. As shown in FIG. 6, Exoskeleton 60 would be able to accommodate different size fingers by means of adjustable fasteners 39, e.g. hook-and-loop. The exoskeleton would maintain exposure of the finger pad so as not to impede tactile sensation.

In one embodiment, endoscopic camera 32 is connected to small battery-powered portable screen module 40 through thin flexible cable 36. Cable 36 transmits digital information from endoscopic camera 32 to screen module 40 and sends power from screen module 40 to camera 32 and LED illuminators 31. Cable 36 can be easily plugged into/unplugged from screen module 40 to allow for multiple disposable finger-mounted video laryngoscopes 10 to be used with a single reusable screen module 40. Additionally, screen module 40 may have an AC power port for charging the battery and wall-powered operation.

Screen module 40 may display overlying crosshairs to help the operator center the glottic opening. Additionally, device 40 might have the ability to record intubations for documentation and teaching purposes. Screen module 40 might contain software that has the ability to recognize the larynx either by image recognition algorithms or by detecting a trans-illuminated light signal sent externally from the patient's anterior neck. In such an embodiment, an audio and/or visual alarm would alert the operator that the target is in view.

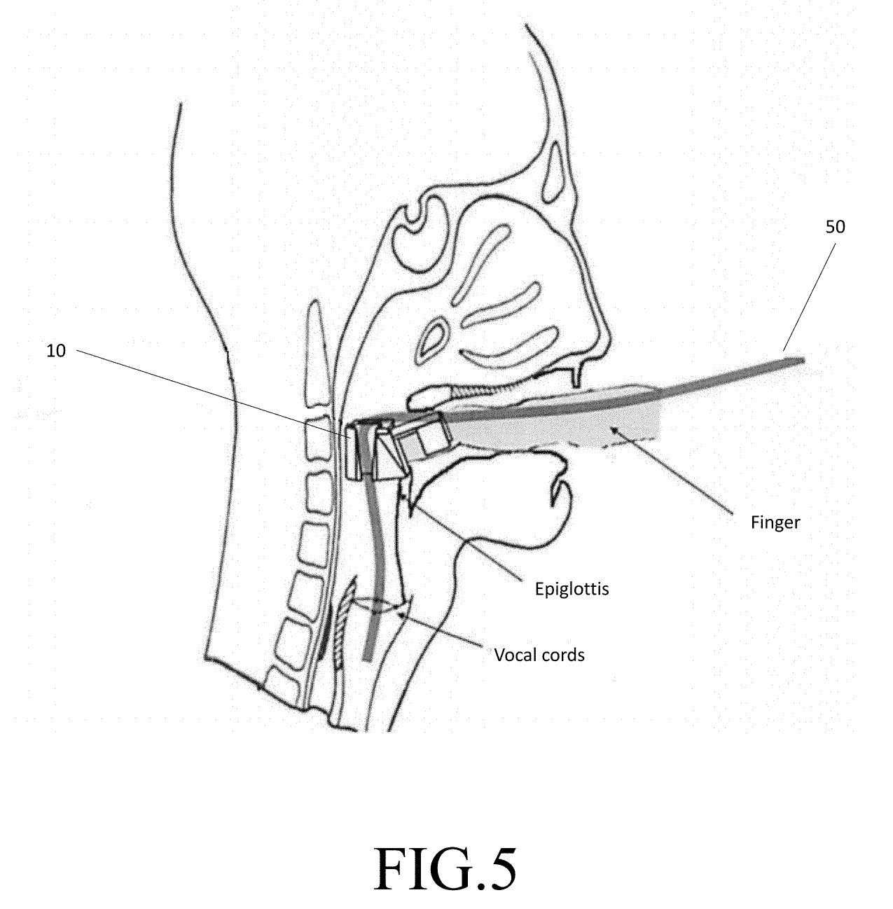

Turning now to an exemplary use of laryngoscopes 10, FIG. 5 shows laryngoscopes 10 on a wearer's finger inserted into the trachea with introducer 50 being inserted past the vocal cords.

In one embodiment, and exemplary list of steps includes, but is not limited to a first step of finger condom 38 and its associated finger-mounted laryngoscope 10 being donned onto the operator's finger. Endotracheal tube introducer 50 is loaded into introducer conduit 34. Thereafter, the user's finger and overlying condom 38 with finger-mounted laryngoscope 10 are inserted into the patient's mouth. The midline of the dorsal surface of tongue is traced by the finger pad until the epiglottis is palpated. The finger is flexed/extended to obtain optimal visualization of the larynx on screen 40 via connected camera 32.

In the next step, endotracheal tube introducer 50 is advanced into the trachea under visualization, and the endotracheal tube introducer 50 is dislodged from the finger-mounted laryngoscope 10 via slit 37. Next, an endotracheal tube is inserted through the rear of inducer 50 and railroaded down the path of introducer 50 into the trachea. Introducer 50 is removed from the endotracheal tube, and finger-mounted video laryngoscope 10 is removed from the mouth.

In another embodiment, an alternative operation of device 10, particularly applicable to nasal and neonatal intubations, is to pass the endotracheal tube into the trachea without using endotracheal tube introducer 50. In this arrangement, finger condom 38 and its associated finger-mounted laryngoscope 10 are donned onto the operator's finger and then the finger and overlying condom 38 with finger-mounted laryngoscope 10 are inserted into the patient's mouth. Next, the midline of the dorsal surface of tongue is traced by the finger pad until the epiglottis is palpated, and the finger is flexed/extended to obtain optimal visualization of the larynx on screen 40. In this alternative, an endotracheal tube is directly introduced into the mouth or nose and manipulated with the index finger so it passes directly into the trachea. The finger-mounted video laryngoscope 10 is then removed from the mouth. Such a method makes use of camera 32 but is faster and does not require the introducer 50 and could be used for example by more experienced providers. In a third embodiment, a third operation of device 10 is to pass endotracheal tube introducer 50 through introducer conduit 34 without visualization of the larynx. This would be employed under conditions when visualization is impossible such as copious vomitus/secretions/blood in the pharynx or tumors that block the airway.

While only certain features of the invention have been illustrated and described herein, many modifications, substitutions, changes or equivalents will now occur to those skilled in the art. It is therefore, to be understood that this application is intended to cover all such modifications and changes that fall within the true spirit of the invention.

What is claimed:

1. A medical device comprising:
a laryngoscope, wherein said laryngoscope has
a finger portion;
a camera; and
introducer conduit,
wherein said camera and said introducer conduit are configured to optimize visualization of a larynx of a patient when inserted into the patient's pharynx.

2. The medial device as claimed in claim 1, wherein said finger portion has a curved fingernail portion, two flexible finger tabs and a recessed area for a finger-tip of user.

3. The medial device as claimed in claim 1, wherein said camera has downward viewing angle perpendicular to said finger portion, and wherein said camera is fitted with peripheral LED lights.

4. The medial device as claimed in claim 3, wherein said camera and LED lights are connected to an electronic device with a screen.

5. The medical device as claimed in claim 1, wherein said finger portion includes a finger condom.

6. The medical device as claimed in claim 1, wherein said finger portion includes an exoskeleton.

7. The medial device as claimed in claim 1, wherein said introducer conduit is slightly larger than an outer diameter of an introducer to be fit therein, sufficient for a friction fit between the two.

8. The medial device as claimed in claim 1, wherein said introducer conduit is curved sufficiently to provide a friction fit with an introducer to be fit therein.

9. The device as claimed in claim 1, wherein said introducer conduit has a flexible diaphragm and/or flexible tabs that friction fit against an introducer to be fit therein.

10. The device as claimed in claim 1, wherein said introducer conduit has a slit such that a lateral force on an introducer fit therein would dislodge it from said introducer conduit.

11. The device as claimed in claim 10, wherein a proximal end of slit has a wider aperture than a distal end of said slit to facilitate dislodgement of said introducer from said introducer conduit.

12. The device as claimed in claim 1, wherein said camera and said introducer conduit are substantially perpendicular to said finger portion, and parallel to one another.
